(19) United States
(12) Patent Application Publication
(10) Pub. No.: US 2024/0277315 A1
(43) Pub. Date: Aug. 22, 2024

(54) WEARABLE ULTRASOUND DEVICE

(71) Applicant: X Development LLC, Mountain View, CA (US)

(72) Inventors: Alexander Martin Zoellner, Los Gatos, CA (US); Ningrui Li, Palo Alto, CA (US)

(21) Appl. No.: 18/444,380

(22) Filed: Feb. 16, 2024

Related U.S. Application Data
(60) Provisional application No. 63/446,652, filed on Feb. 17, 2023.

(51) Int. Cl. A61B 8/00; A61B 8/08

(52) U.S. Cl. CPC A61B 8/4236; A61B 8/4254; A61B 8/4281; A61B 8/4477; G01S 15/8934; A61B 8/483; A61B 8/565; A61B 2562/164

(57) ABSTRACT

The present disclosure relates to a wearable ultrasound device comprising a flexible body comprising a plurality of layers, a shape sensor integrated into a first layer of the flexible body, and an ultrasound transducer coupled to the shape sensor. The ultrasound transducer is configured for capturing images of a subject, and the shape sensor is configured for determining a location of the ultrasound transducer relative to the flexible body.

### CROSS-REFERENCE TO RELATED APPLICATIONS

This application claims priority of U.S. Provisional Application Ser. No. 63/446,652 filed Feb. 17, 2023. The contents of the prior application is incorporated herein by reference in its entirety.

### TECHNICAL FIELD

The present disclosure relates to medical devices, and in particular, to medical devices used for ultrasound imaging.

### BACKGROUND

Medical imaging devices, such as computed tomography scans and magnetic resonance imaging, provide medical practitioners with information to diagnose, monitor, and treat medical conditions. Medical imaging devices primarily recreate images or other types of representations such as models of parts of the body.

Conventional medical devices are expensive to operate and can provide measurements with poor-fidelity, leading to inaccurate medical diagnoses and very little insight in the growth or recovery of examined tissue. Poor measurement acquisition can be due to the inconsistent techniques applied from operator to operator. Medical devices provide a narrow perspective of examined tissue and often overlook other sensing technologies that can improve the overall fidelity of the acquired images. Additionally, the fidelity of the acquired data by a medical device is highly dependent on the operator of the medical device, which can range from a layperson with minimal training, and therefore low-cost for imaging, to a highly specialized medical professional with extensive training, and therefore a highly expensive cost for obtaining medical images. Ultrasound imaging devices are low-cost, portable, and provide images in real-time though generally are operated by a skilled user, usually with professional training.

### SUMMARY

A wearable ultrasound device of the present disclosure utilizes a shape sensor and an ultrasound transducer to take multiple images and compound the images to create a three-dimensional scan of a body part. The ultrasound device is capable of taking three-dimensional images from multiple scans.

In one aspect, the present disclosure provides a wearable ultrasound device including a flexible body including a plurality of layers, a shape sensor integrated into a first layer of the flexible body; and an ultrasound transducer coupled to the shape sensor, wherein the ultrasound transducer is configured for capturing images of a subject, and the shape sensor is configured for determining a location of the ultrasound transducer relative to the flexible body.

Examples may include one or more of the following features. The wearable ultrasound device can be disposed in a second layer adjacent to the first layer. The third layer can be a pliant buffer layer. The wearable ultrasound device may include a fourth layer adjacent to the second layer, the fourth layer may include a coupling gel that can be configured for placement against a body of the subject. The fourth layer may include a hydrogel and an adhesive for securably coupling the flexible body to the subject. The shape sensor can be a network of strain gauges. The shape sensor can be a fiber optic. The shape sensor and the ultrasound transducer can be disposed in the first layer of the flexible body. The wearable ultrasound device of any one through 9, may include a controller which may include one or more processors, the one or more processors configured to generate one or more images based on data received by the ultrasound transducer and a location of the ultrasound transducer. The wearable ultrasound device may include a wireless communication device coupled to the controller. The wearable ultrasound device may include a plurality of ultrasound transducers integrated with the flexible body. The plurality of ultrasound transducers can be arranged in an array. The flexible body can have a thickness in a range of approximately 0.1 mm to approximately 20 mm.

In another aspect, the present disclosure provides a wearable ultrasound device including a flexible body arranged to mold against a body of a subject, an ultrasound transducer integrated with the flexible body, the ultrasound transducer configured to capture one or more images of a body part of the subject, and a strain gauge coupled to the ultrasound transducer, the strain gauge configured to determine a location of the ultrasound transducer relative to the flexible body.

Examples may include one or more of the following features. The wearable ultrasound device may include a plurality of ultrasound transducers integrated with the flexible body. The plurality of ultrasound transducers can be arranged in an array. The wearable ultrasound device may include a controller coupled to the strain gauge and the ultrasound transducer, the controller configured to process, using one or more processors, the location of the ultrasound transducer and one or more images of the body part captured by the ultrasound transducer. The controller can be configured to create a three-dimensional scan of the body part using the one or more images and the location of the ultrasound transducer. The flexible body may include a first layer adjacent to a second layer, where the strain gauge can be integrated into the first layer and a buffer can be integrated with the second layer. The ultrasound transducer can be embedded in the first layer. The ultrasound transducer can be integrated in a third layer adjacent to the second layer. The wearable ultrasound device may include a fiber optic. The flexible body can have an outer layer which may include an adhesive adapted for secure attachment to the body of the subject. The outer layer may include an ultrasound gel.

### Definitions

As used herein, the term “about” means+/−10% of any recited value. As used herein, this term modifies any recited value, range of values, or endpoints of one or more ranges.

As used herein, the terms “top,” “bottom,” “upper,” “lower,” “above,” and “below” are used to provide a relative relationship between structures. The use of these terms does not indicate or require that a particular structure must be located at a particular location in the apparatus.

Some examples may be described using the expression “coupled” and “connected” along with their derivatives. For example, some arrangements may be described using the term “coupled” to indicate that two or more elements are in direct physical or electrical contact. The term “coupled,” however, may also mean that two or more elements are not in direct contact with each other, but yet still co-operate or interact with each other. The examples described herein are not limited in this context.

Other features and advantages of the present disclosure will be apparent from the following detailed description, figures, and claims.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is an example schematic diagram of using a wearable ultrasound device in accordance with the teachings of the present disclosure;

FIG. 2A is a cross-sectional view of the wearable ultrasound device of FIG. 1, taken at A, in accordance with the teachings of the present disclosure;

FIG. 2B is an example schematic diagram of using a wearable ultrasound device having and ultrasound transducer array in accordance with the teachings of the present disclosure; and

FIG. 3 is a cross-sectional view of another example of a wearable ultrasound device constructed in accordance with the teachings of the present disclosure.

### DETAILED DESCRIPTION

A wearable ultrasound device of the present disclosure utilizes a shape sensor and an ultrasound transducer to take multiple images and compound the images to create a three-dimensional scan of a body part.

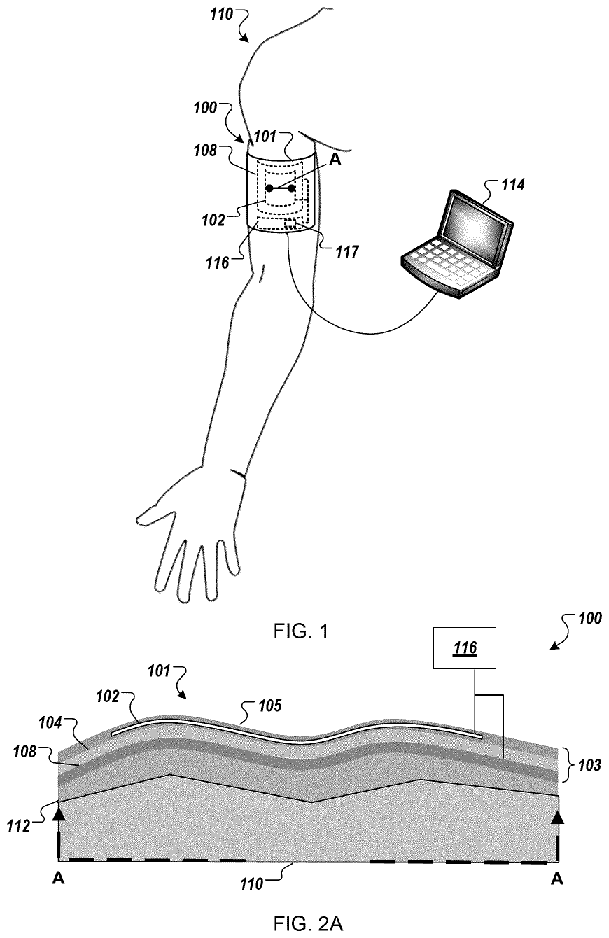

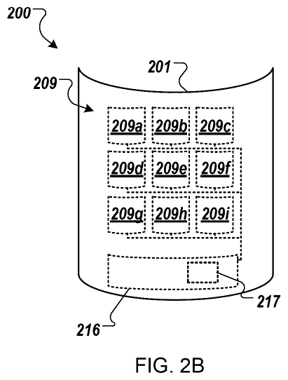

Referring now to FIGS. 1 and 2, a wearable ultrasound device 100 is shown affixed to an arm of a patient 110 to image a portion of the arm, such as the bicep over which the wearable ultrasound device 100 is placed. The device 100 is communicatively coupled to a user device 114 that receives imaging data from the wearable ultrasound device 100 on the patient 110. Generally, the ultrasound device 100 may be used to acquire and/or transmit medical data, e.g., measurements and/or images, from the patient, by applying the ultrasound device 100 to a region of an appendage (e.g., arm, leg) or body part (e.g., abdomen) to be examined, e.g., an examination region. The ultrasound device 100 may be used to capture medical data from a stationary examination region, such as an appendage, and may also be used to capture medical data from moving examination regions, such as a respirating chest or a beating heart.

The ultrasound device is used to capture high-resolution images and/or video of the examination region through acoustic data generated from one or more ultrasound transducers. An examination region is a physical location or area on the body of the subject. In some examples, the examination region includes multiple organs to be imaged, e.g., an abdomen can be examined to look at muscle tissue and organs that include intestines, liver, etc. For example, the ultrasound device 100 may acquire data related to the examination region using one or more sensors to capture an image of the examination region using the generated data. In some examples, the captured image is a two-dimensional (2D) image depicting a ‘slice’ of the examination region across a plane. In another example, the constructed image is a three-dimensional (3D) image, such as a virtual tomograpic image, of the examination region.

The ultrasound device 100 has a flexible body 101 that is moldable to the examination region of the patient 110. The body 101 includes different layers 103 integrating a flexible ultrasound transducer 108, a shape sensor 102, and a controller 116. The ultrasound transducer 108, shape sensor 102, and controller 116 are shown in dashed lines in FIG. 1 to represent being enclosed or otherwise embedded in the body 101.

The ultrasound device 100 is affixed to the patient 110 and captures images of the examination region. To capture the images, the ultrasound transducer 108 generates sound waves in the ultrasound spectrum (e.g., ≥20 kHz) directed into the examination region. The sound waves reflect from one or more internal body structures of the subject such as tendons, muscles, joints, blood vessels, and internal organs and the ultrasound transducer 108 receives the reflected sound waves and generates data based on the reflected waves. The ultrasound transducer 108 communicates the data to the controller 116, which generates images based on the data, thereby capturing the images of the examination region.

Generally, the ultrasound device 100 captures images of the examination region, the volume of which corresponds with the area of the ultrasound transducer 108, the imaging depth, and the ultrasound beam shape. In some examples, the examination region which can have an area in a range from approximately 10 square centimeters (sqcm) to approximately 500 sqcm (e.g., 50 sqcm to 500 sqcm, 100 sqcm to 500 sqcm, 200 sqcm to 500 sqcm, 50 sqcm to 200 sqcm, 10 sqcm to 200 sqcm, 10 sqcm to 100 sqcm, 10 sqcm to 50 sqcm, or 100 sqcm to 200 sqcm).

The shape sensor 102 is integrated into a first layer 105 of the ultrasound device 100. The shape sensor 102 generates data based on the shape and position of the shape sensor 102. As the shape sensor 102 is disposed in the layer 105 near the ultrasound transducer 108, the shape of the shape sensor 102 generally corresponds with the shape of the ultrasound transducer 108. The shape sensor 102 generates shape data based on a relative spatial positions of the shape sensor 102 as well as axial, bending, shear, and/or torsional strain applied to the shape sensor 102. The shape sensor 102 measures relative position within the sensor 102 and the controller 116 correlates this data to relative position information of the elements of the ultrasound transducer 108. In further examples, the ultrasound device 100 includes a 6 degree-of-freedom (DOF) spatial positioning system such as a gyro or accelerometer.

The shape sensor 102 generates shape data with sufficient resolution such that when the shape data is processed along with the acoustic data from the ultrasound transducer 108, the controller 116 generates high resolution images of the subject. The ultrasound device 100 generates images having resolution of about 1 mm. Higher resolutions (e.g., about 10 μm) are possible with higher frequencies or ultrasound super resolution techniques. Broadly, the ultrasound device 100 can be configured to achieve resolutions in a range from approximately 10 μm to approximately 5 mm (e.g., from 20 μm to 5 mm, from 50 μm to 5 mm, from 100 μm to 5 mm, from 200 μm to 5 mm, from 1 mm to 5 mm, from 2 mm to 5 mm, from 10 μm to 2 mm, from 10 μm to 1 mm, from 10 μm to 200 μm, from 10 μm to 100 μm, from 10 μm to 50 μm, from 10 μm to 20 μm, from 50 μm to 200 μm, from 100 μm to 200 μm, or from 50 μm to 1 mm).

The controller 116 includes at least one processor 117 and is communicatively coupled to the shape sensor 102 and the ultrasound transducer 108. In general, the controller 116 is integrated into the same layer as the ultrasound transducer 108, e.g., the top layer. The controller 116 receives shape data from the shape sensor 102 and acoustic data from the ultrasound transducer 108. Collecting the shape and acoustic data and processing them simultaneously increases the resolution and reduces error, e.g., noise, in captured medical data produced by the controller 116. The controller 116 captures the medical data as 2D and/or 3D images, e.g., scans or slices, of the examination region from the received data. As one example, the controller 116 generates a 2D planar image of a portion of the examination region under the ultrasound device 100. As another example, the controller 116 generates a series of 2D planar images of the area and generates a 3D tomographic image based on the 2D series. In other examples, the controller 116 may be integrated into a different layer than the ultrasound transducer.

The ultrasound device 100 enables captured medical data to be viewed and/or manipulated by a user. In such an example, the controller 116 generates a series of 2D or 3D images and generates a time-series of images, e.g., a video, of the data for viewing on a connected device, such as the user device 114. The controller 116 can generate visualizations based on generated data and communicate the visualizations following capture, or the controller 116 can process the generated data in real-time, e.g., in parallel, to communicate the visualizations in real-time to the user device 114.

In general, the controller 116 is configured to communicate with external processing devices, e.g., computers, through wired or wireless communication. In the example of FIG. 1, the ultrasound device 100 is in wired communication with the user device 114, which receives the image data compiled by the controller 116. The user device 114 includes a display, storage, processor and various controls such that a user interacting with the user device 114 can view, manipulate, or store the images received from the controller 116 and presented to the user.

The user device 114 is shown in a wired connection to the ultrasound device 100, though in other examples, the ultrasound device 100 can be wirelessly connected to the user device 114, such as via Bluetooth™, or radio communication (e.g., Wi-Fi). In some examples, the controller 116 provides the data from the shape sensor 102 and the ultrasound transducer 108 to the user device 114 for processing into 2D or 3D images or videos.

In the example of FIG. 2, the ultrasound device 100 includes a pliant buffer layer 104 separating the shape sensor layer 105 and the transducer layer 108. The buffer layer 104 allows the layers 103 of the flexible body 101 to move relative to each other so that the device 100 can mold to the subject. Though in other examples, the shape sensor 102 and ultrasound transducer 108 layers may be adjacent or embedded in the same layer. The ultrasound device 100 is thin to facilitate portability and application ease on the patient 110. In general, the thickness of the ultrasound device 100 is in a range from approximately 0.1 mm to 2 approximately 0 mm (e.g., 0.5 mm to 20 mm, 1 mm to 20 mm, 5 mm to 20 mm, 10 mm to 20 mm, 0.1 mm to 15 mm, 0.1 mm to 10 mm, 0.1 mm to 5 mm, 0.1 mm to 1 mm, 0.1 mm to 0.5 mm, 0.5 mm to 10 mm, 0.5 mm to 5 mm, 1 mm to 10 mm, or 1 mm to 5 mm).

The ultrasound transducer 108 includes a flexible array of ultrasound transceiver elements that create the transducer layer 108. In other examples, the transducer layer 108 may include a material and the array embedded or integrated into the material. Examples of the ultrasound transducer 108 include a piezoelectric micromachined ultrasonic transducers (PMUT), a capacitive micromachined ultrasonic transducer (CMUT), or an array of bulk piezoelectric elements, such as piezoelectric-ceramic elements (e.g., a PZT or single-crystal PMN-PT).

The number of ultrasound transceiver elements in the ultrasound transducer 108 can be in a range from approximately 16 to approximately 100,000 elements (e.g., from 16 to 3600 elements, from 16 to 1024 elements, from 16 to 512 elements, from 16 to 64 elements, from 64 to 512 elements, from 64 to 1024 elements, from 64 to 8000 elements, from 512 to 1024 elements, from 512 to 8000 elements, from 1024 to 8000 elements, from 3600 to 8000 elements, from 8,000 to 20,000 elements, from 8,000 to 50,000 elements, from 20,000 to 50,000 elements, from 20,000 to 50,000 elements, from 10,000 to 50,000 elements, from 20,000 to 100,000 elements, or from 50,000 to 100,000 elements).

In some examples, the ultrasound device 100 includes multiple, e.g., more than one, ultrasound transducers 108 in the same layer, e.g., two, three, four, or more ultrasound transducers 108. The number of ultrasound transducers 108 depends on the overall area of the ultrasound device 100 and the individual size and shape of the ultrasound transducers 108. For instance, two ultrasound transducers 108 arranged adjacent to each other in the same layer allow differential imaging of two examination regions beneath the ultrasound device 100. Particularly, one ultrasound transducer 108 may generate acoustic data from a first area of the region, while the second ultrasound transducer 108 may generate acoustic data from a second area.

Referring to FIG. 2B, an ultrasound device 200 is shown having a body 201 integrating a flexible ultrasound transducer array 209, a shape sensor, and a controller 216 having a processor 217. The ultrasound transducer array 209 and controller 216 are shown in dashed lines in FIG. 2B to represent being enclosed or otherwise embedded in the body 201, though the shape sensor is not shown for ease of visualization of the ultrasound transducer 209. The ultrasound transducer array 209 includes a 3×3 array of ultrasound transducers 209a-209i, each connected to the controller 216. In general, the body 101, controller 116, shape sensor 102, ultrasound transducer 108, and processor 117 can provide the components of the ultrasound device 200 of FIG. 2, including the body 201, controller 216, shape sensor (unlabeled), the ultrasound transducers 209a-209i, and processor 217.

In examples in which the ultrasound device 100 includes multiple ultrasound transducers 108, the transducers 108 can be arranged in an n by m tiled array (e.g., a 1×2, a 2×2, a 1×4, a 2×4, a 3×4, a 4×4, a 1×8, a 2×8, a 3×8, or a 4×8 tiled array). Individual ultrasound transducers 108 having similar or identical shape can be arranged adjacently such that the medical data produced are partially overlapping and cover a larger examination region. The ultrasound device 100 can include individual ultrasound transducers 108 in a range from 2 to 32 arranged in a tiled array (e.g., from 2 to 4, from 2 to 8, from 2 to 16, from 2 to 24, from 4 to 8, from 4 to 16, from 4 to 24, from 4 to 32, from 8 to 16, from 8 to 24, from 8 to 32, from 16 to 24, from 16 to 32, from 24 to 32, from 4 to 24, from 8 to 24, from 16 to 24, from 2 to 16, from 2 to 8, from 4 to 8, from 4 to 16, from 4 to 24, from 8 to 16, or from 8 to 24).

An example of the shape sensor 102 is an array of strain gauge sensors. Each strain gauge sensor of the array generates data along one or more dimensions of the shape sensor 102. Another example of the shape sensor 102 is a fiber Bragg grating (FBG) strain sensor which is a segment of optical fiber embedded within the shape sensor 102 layer of the ultrasound device 100. Changes in the shape of the segment are indicative of the strain applied. An FBG strain sensor provides a higher-resolution solution than the strain gauge array, while a strain gauge array provides a lower-cost solution than the FBG sensor.

In further examples, the ultrasound device 100 includes multiple layers of shape sensor 102, which can have different orientations between layers. Different orientations between layers increase the spatial and angular resolution of the shape data generated by the ultrasound device 100. For example, FBG strain sensors oriented orthogonally in the ultrasound device 100 generate shape data along dimensions at a right angle. In another example, multiple shape sensors 102 oriented at between 30° and 60° of each layer (e.g., 45°) generate redundant data along orthogonal directions of the ultrasound device 100. In another example, the ultrasound device 100 includes both an array of strain gauge sensors and one or more FBG sensors which can be embedded in the same layer, or different layers.

The shape sensor 102 and the ultrasound transducer 108 are flexible and forces are generated between the shape sensor 102 and ultrasound transducer 108 as the relative positioning between layers changes as the ultrasound device 100 changes shape. The forces, e.g., stress, or strain, arise due to differences in rigidities, thicknesses, and positioning of adjacent layers. The buffer layer 104 is positioned between the shape sensor 102 and the ultrasound transducer 108 and deforms based on differences in flexural positioning between the sensor 102 and transducer 108 to reduce the forces generated. The buffer layer 104 is manufactured from a soft, flexible material such as hydrogel, silicon, natural rubber, synthetic rubber (e.g., neoprene), or fabric, and can be a woven, solid, or foam layer.

In another example, the ultrasound device 100 includes optical markers displayed on world-facing surfaces. The optical markers can be imaged, e.g., with a camera, or cell phone, and additional relative shape data of the ultrasound device 100 generated from the imaged positions of the optical markers. The shape data generated using the optical markers can be communicated to the controller 116 for processing with the shape data generated by the shape sensor 102.

A coupling gel 112 is shown separating the ultrasound transducer 108 and the patient 110. The coupling gel 112 enables the sound to be transmitted efficiently into the patient 110 through acoustic impedance matching. In general, the coupling gel 112 is a water-based gel to facilitate ultrasound transmission into the patient 110, e.g., an ultrasound gel. In one example, the patient 110, or an assisting user, e.g., a medical technician, applies the coupling gel 112 to the examination region and applies the ultrasound device 100 to the coupling gel 112.

The layers of the ultrasound device 100 are coupled together such that motion in one layer induces motion in the coupled layers. The layers can be adhered together, or sealed together in the body 101 that constrains the motion of the layers.

In another example, ultrasound device 100 includes an outer layer 112, separating the ultrasound transducer 108 from the subject 110. The outer layer includes a coupling gel 112, which may be a hydrogel that can be affixed to the user-facing side of the ultrasound transducer 108 to adhere to the patient 110 when the ultrasound device 100 is applied to the examination region. The coupling gel 112 can also include an adhesive agent to increase the adhesion between the coupling gel 112 and the patient 110. In one example, the gel 112 is a commercially-available silicone-based dry coupler (Sonemat, UK). In a further example, the gel 112 is a hydrogel-based dry coupler.

In further examples, the ultrasound device 100 is functional to perform non-medical imaging such as non-destructive testing of constructions, materials, or devices. The ultrasound device 100 is temporarily affixable to any surface to be imaged, e.g., 2D imaged, or 3D imaged. In some examples, this includes in a construction setting, a maintenance setting, a quality control setting, or a materials testing setting.

In more examples, the ultrasound device 100 includes sensors that generate medical data in addition to the captured images. The generation of additional medical data may beneficially increase the accuracy of the images generated by the ultrasound device 100. The ultrasound device 100 may include a thermometer for determining a temperature. The ultrasound device 100 may include an accelerometer for generating motion data. The ultrasound device 100 may include a galvanic skin response (GSR) stress sensor.

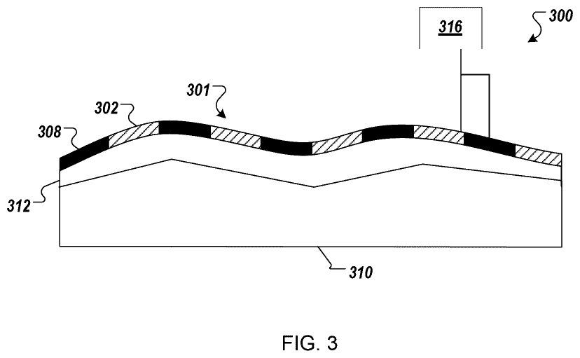

In a further example, the shape sensor 102 and the ultrasound transducer 108 can occupy the same layer of the ultrasound device 100. Referring to FIG. 3, a different example ultrasound device 300 has a flexible body 301 including a shape sensor 302 and an ultrasound transducer array 308 that reside in the same layer. In this example, the transducer array 308 is an array of ultrasound transducers and the shape sensor 302 is a network of positioning sensors. The ultrasound array 308 and the shape sensor 302 are interwoven, e.g., interleaved, such that in a longitudinal direction of the ultrasound device 300, the individual transducers of the ultrasound array 308 and sensors of the shape sensor 302 alternate. The interwoven configuration may help reduce error in correlating the position data to the positions of the transducers of the transducer array 308. The ultrasound device 300 is affixed to the patient 310 with a hydrogel layer 312 and a controller 316 is connected to the shape sensor 302 and the transducer array 308, which can be provided by the gel 112 and controller 116 of FIG. 1, respectively.

As one example of a use of the ultrasound device 100, a subject, e.g., patient 110, may receive the ultrasound device 100 from a medical distributor, such as a pharmacy, in the mail, or at medical professional office. The subject connects the ultrasound device 100, 300, e.g., through Bluetooth or USB, to a computing device, such as a laptop or phone, e.g., user device 114. The subject applies the ultrasound device 100, 300, which may be in the form of a patch or bandage, to an examination region on an examination areas as instructed by their medical provider or through simple instructions on the package. The connected computing device 114 may be communicatively connected to the healthcare provider through a network, e.g., the internet, in a virtual medical environment, such as a remote health visit. The connected computing device 114 can receive the medical data, e.g., the shape sensor data and the transducer data, from the ultrasound device 100, 300 and transmit the medical data to the healthcare provider in attendance during the remote visit. The healthcare provider can read, interpret, view, or manipulate the results in real time and/or provide further instructions to the subject, e.g., instructions to move the patch, or instructions to remain stationary for a duration. In some examples, the subject can capture the medical data using the ultrasound device 100, 300 without being attended by a healthcare professional, e.g., alone. In such examples, a medical professional can review the medical data and respond to the patient at a later time.

The ultrasound devices 100, 300 disclosed herein may facilitate detailed imaging in any environment, such as the situation described above, without the need for complex or expensive training, thereby democratizing ultrasound technology. This applies to medical environments, or non-destructive testing.

The ultrasound devices 100, 300 may facilitate improved communication between medical professionals and users of the device, e.g., telehealth visits. The user may collect detailed medical imaging information and communication the information to a medical professional at another location for further interpretation and diagnosis.

The ultrasound devices 100, 300 may reduce costs and training related to taking 2D or 3D ultrasound imaging for medical institutions. The device may be rapidly applied over an examination region by an untrained user and the imaging initiated on a local or remote device.

The ultrasound devices 100, 300 may generate images and real-time video of the imaged examination region which facilitates rapid interpretation and diagnoses of a wide variety of medical disorders. This beneficially decreases the time required to provide diagnosis and the time to initiate treatment, which increases the efficacy of medical intervention.

The ultrasound devices 100, 300 may be programmed to use, or be communicatively coupled to an external device which uses, a computing model to process data from one or more sensors of the wearable ultrasound devices 100, 300. The computing model may include a machine learning neural network, a physical simulation, a computational model, or some combination therein. The computing model may reconstruct a surface representation or multi-dimensional representations of the examination region of patient 110 by combining position measurements (e.g., from the shape sensor 102) with the acoustic data received from the flexible ultrasound transducer 108. In some examples, the computing model can extrapolate future measurement data from the measurements of sensors in the ultrasound devices 100, 300, in which the future measurement data describe the progression (e.g., recovery, spread of disease) of the examination region. In another example, the computing model can interpolate measurement data from the measurements of sensors in the ultrasound devices 100, 300, in which progression is described based on sparsely or intermittently collected medical data.

This specification uses the term “configured” in connection with systems and computer program components, such as the ultrasound device 100, 300, user device 114, or controller 116. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by a data processing apparatus, such as controller 116, cause the apparatus to perform the operations or actions.

The term “data processing apparatus,” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array), complementary metal-oxide semiconductor (CMOS) microcontroller, printed circuit board (PCB), flexible PCB, or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers, such as a laptops, personal computers, personal digital assistants, cell phones, or tablets, suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer, e.g., user device 114, having a display device, e.g., a CRT (cathode ray tube) or LCD (liquid crystal display) monitor, for displaying information to the user and a keyboard and a pointing device, e.g., a mouse or a trackball, by which the user can provide input to the computer.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

While this specification contains many specific implementation details, these should not be construed as limitations on the scope of any disclosure or of what may be claimed, but rather as descriptions of features that may be specific to particular examples of particular disclosures. Certain features that are described in this specification in the context of separate examples can also be implemented in combination in a single example. Conversely, various features that are described in the context of a single example can also be implemented in multiple examples separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the examples described herein should not be understood as requiring such separation in all examples, and it should be understood that the described program components and systems can generally be integrated together in a single product or packaged into multiple products.

Particular examples of the subject matter have been described. Other examples are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A wearable ultrasound device comprising:
a flexible body comprising a plurality of layers;
a shape sensor integrated into a first layer of the flexible body; and
an ultrasound transducer coupled to the shape sensor, wherein the ultrasound transducer is configured for capturing images of a subject, and the shape sensor is configured for determining a location of the ultrasound transducer relative to the flexible body.

2. The wearable ultrasound device of claim 1, wherein the ultrasound transducer is disposed in a second layer adjacent to the first layer.

3. The wearable ultrasound device of claim 2, comprising a third layer disposed between the first layer and the second layer, wherein the third layer is a pliant buffer layer.

4. The wearable ultrasound device of claim 3, comprising a fourth layer adjacent to the second layer.

5. The wearable ultrasound device of claim 4, wherein the fourth layer comprises a coupling gel that is configured for placement against a body of the subject.

6. The wearable ultrasound device of claim 1, wherein the shape sensor is a network of strain gauges.

7. The wearable ultrasound device of claim 1, wherein the shape sensor is a fiber optic.

8. The wearable ultrasound device of claim 1, wherein the shape sensor and the ultrasound transducer are disposed in the first layer of the flexible body.

9. The wearable ultrasound device of claim 1, comprising a controller comprising one or more processors, the one or more processors configured to generate one or more images based on data received by the ultrasound transducer and a location of the ultrasound transducer.

10. The wearable ultrasound device of claim 9, comprising a wireless communication device coupled to the controller.

11. The wearable ultrasound device claim 1, comprising a plurality of ultrasound transducers integrated with the flexible body and arranged in an array.

12. The wearable ultrasound device of claim 1, wherein the flexible body has a thickness in a range of approximately 0.1 mm to approximately 20 mm.

13. A wearable ultrasound device comprising:
a flexible body arranged to mold against a body of a subject;
an ultrasound transducer integrated with the flexible body, the ultrasound transducer configured to capture one or more images of a body part of the subject; and
a strain gauge coupled to the ultrasound transducer, the strain gauge configured to determine a location of the ultrasound transducer relative to the flexible body.

14. The wearable ultrasound device of claim 13, wherein the ultrasound transducer comprises a plurality of ultrasound transducers integrated with the flexible body.

15. The wearable ultrasound device of claim 14, wherein the plurality of ultrasound transducers is arranged in an array.

16. The wearable ultrasound device of claim 13, comprising a controller coupled to the strain gauge and the ultrasound transducer, the controller configured to process, using one or more processors, the location of the ultrasound transducer and one or more images of the body part captured by the ultrasound transducer.

17. The wearable ultrasound device of claim 16, wherein the controller is configured to create a three-dimensional scan of the body part using the one or more images and the location of the ultrasound transducer.

18. The wearable ultrasound device of claim 13, wherein the flexible body comprises a first layer adjacent to a second layer, wherein the strain gauge is integrated into the first layer and a buffer is integrated with the second layer.

19. The wearable ultrasound device of claim 18, wherein the ultrasound transducer is embedded in the first layer.

20. The wearable ultrasound device of claim 18, wherein the ultrasound transducer is integrated in a third layer adjacent to the second layer.
